(19) United States
(12) Patent Application Publication
(10) Pub. No.: US 2023/0142793 A1
(43) Pub. Date: May 11, 2023

(54) Reversible Electroporation for Cardiac Defibrillation

(71) Applicant: Mayo Foundation for Medical Education and Research, Rochester, MN (US)

(72) Inventors: Samuel J. Asirvatham, Rochester, MN (US); Paul A. Friedman, Rochester, MN (US)

(21) Appl. No.: 17/912,751

(22) PCT Filed: Mar. 30, 2021
(86) PCT No.: PCT/US2021/024811

§ 371 Date: Sep. 19, 2022

Related U.S. Application Data
(60) Provisional application No. 63/002,060, filed on Mar. 30, 2020.

(51) Int. Cl. A61N 1/05; A61N 1/32

(52) U.S. Cl. CPC A61N 1/0563; A61N 1/327; A61N 1/0587

(57) ABSTRACT

This disclosure describes methods systems, and devices for treating atrial and ventricular fibrillation. For example, this document describes a method of terminating cardiac arrhythmias that includes generating a bipolar pulsed electrical field between a first electrode positioned on a first portion of a heart of a patient and a second electrode positioned on a second portion of the heart of the patient to cause reversible electroporation of myocardial cells of the heart.

### CROSS-REFERENCE TO RELATED APPLICATIONS

This application claims the benefit of U.S. Provisional Application Ser. No. 63/002,060, filed Mar. 30, 2020. The disclosure of the prior application is considered part of (and is incorporated by reference in) the disclosure of this application.

### TECHNICAL FIELD

This disclosure relates to methods and materials for treating cardiac fibrillation. For example, this document relates to methods and devices for delivering reversible electroporation to cardiac tissue to treat cardiac fibrillation.

### BACKGROUND

Sudden cardiac death is a leading cause of mortality, the majority of which is due to ventricular fibrillation. Occurring either as a primary event or secondary event to concomitant cardiac and non-cardiac diseases and events, prevention of this arrhythmia remains rudimentary. While defibrillators and anti-arrhythmics provide an element of protection in select cases, sudden cardiac death remains a major worldwide health problem. In addition, a major drawback of defibrillators is the severe lifestyle-limiting pain that results from shocks provided by the defibrillator to stop ventricular fibrillation. As a result, some patients elect to forgo this therapy, or elect to turn off their defibrillators and remain at risk of sudden death.

In addition, atrial fibrillation is the most common cardiac rhythm disorder affecting patients. However, because atrial fibrillation is not typically a life-threatening condition and the pain associated with the use of traditional defibrillators is severe, defibrillators have not been routinely utilized for treating atrial fibrillation.

Electroporation is a technique that uses high voltage to non-thermally introduce multiple nanopores within the cells' wall, specifically within the lipid bilayer of the cell membranes as a result of the change in electrical field. Depending on the voltage and frequency of pulsations used, these pores can be reversible (i.e., increase the permeability of these cell to chemotherapeutic agents) and or irreversible (i.e., trigger cell death by the process of apoptosis or necrosis). Given the different composition of each cell-type membrane, electroporation can allow for a differential effect on different tissues.

### SUMMARY

This disclosure describes methods and materials for treating atrial and ventricular fibrillation. For example, this document describes methods and devices to deliver reverse electroporation to cardiac tissue to treat atrial and ventricular fibrillation.

In one aspect, this disclosure is directed to a method of terminating cardiac arrhythmias that includes generating a bipolar pulsed electrical field between a first electrode positioned on a first portion of a heart of a patient and a second electrode positioned on a second portion of the heart of the patient to cause reversible electroporation of myocardial cells of the heart.

Embodiments can include one or more of the following features in any combination.

In certain embodiments, the method further includes monitoring the electrical activity of the heart of the patient to determine the presence of a heart arrhythmia, wherein the pulsed electrical field is generated in response to detecting the presence of the heart arrhythmia

In some embodiments, generating a pulsed electrical field includes generating a plurality of electrical pulses between the first electrode and the second electrode with a pulse width in a range of 1 nanosecond to 300 microseconds.

In certain embodiments, an electrical potential of the pulsed electrical field ranges from 0.2 microCoulombs to about 30 milliCoulombs

In some embodiments, generating a pulsed electrical field between the first electrode positioned on the first portion of the heart of the patient and the second electrode positioned on the second portion of the heart of the patient: porates cell membranes of myocardial cells of the heart; and increases cardiac ion channel conduction within the myocardial cells.

In certain embodiments, the pulsed electrical field passes through a critical mass of myocardial tissue of the heart of the patient.

In some embodiments, the first portion of the heart includes a first portion an epicardial surface of the heart or a first portion of an endocardial surface of the heart; and the second portion of the heart includes a second portion an epicardial surface of the heart or a second portion of an endocardial surface of the heart.

In certain embodiments, the first portion of the heart includes an endocardial surface of an atrium of the heart of the patient; the second portion of the heart includes an epicardial surface of the atrium of the heart of the patient; and generating the bipolar pulsed electrical field affects atrial conduction.

In some embodiments, the first portion of the heart includes an endocardial surface of a ventricle of the heart of the patient; and the second portion of the heart includes an epicardial surface of the ventricle of the heart of the patient, wherein generating the bipolar pulsed electrical field affects ventricular conduction.

In certain embodiments, the method further includes monitoring the electrical activity of the heart of the patient following the generation of the bipolar pulsed electrical field; determining a heart arrhythmia is present; and in response to determining a heart arrhythmia is present, generating another bipolar pulsed electrical field.

In some embodiments, the method further includes positioning the first electrode on the first portion of the heart of the patient; and positioning the second electrode on the second portion of the heart of the patient.

In certain embodiments, the first electrode includes a first portion of an inductor coil; the second electrode includes a second portion of the inductor coil; and positioning the first electrode on the first portion of the heart of the patient includes positioning the first portion of the inductor coil against the first portion of the heart; and positioning the second electrode on the second portion of the heart of the patient includes positioning the second portion of the inductor coil against the first portion of the heart.

In some embodiments, the first electrode and the second electrode are attached to at least one mesh; positioning the first electrode on the first portion of the heart of the patient includes positioning the at least one mesh on a surface of the heart to position the first electrode against the first portion of the heart; and positioning the second electrode on the second portion of the heart of the patient includes positioning the at least one mesh on a surface of the heart to position the second electrode against the second portion of the heart.

In certain embodiments, the first electrode is attached to a first mesh; the second electrode is attached to a second mesh; positioning the first electrode on the first portion of the heart of the patient includes positioning the first mesh on a first surface of the heart; and positioning the second electrode on the second portion of the heart of the patient includes positioning the second mesh on a second surface of the heart.

In another aspect, a system includes a first electrode; a second electrode; a generator electrically coupled to the first electrode and the second electrode, the generator being configured to provide pulsating direct current to the first electrode and the second electrode; and at least one attachment device configured to position the first electrode at a first portion of a heart of a patient and position the second electrode at a second portion of the heart of the patient.

Embodiments can include one or more of the following features in any combination.

In some embodiments, the at least one attachment device includes a biocompatible mesh; and the first electrode and the second electrode are coupled to the biocompatible mesh.

In certain embodiments, the at least one attachment device includes: a first biocompatible mesh, the first electrode being coupled to the first biocompatible mesh; and a second biocompatible mesh, the second electrode being coupled to the second biocompatible mesh.

In some embodiments, the attachment device includes an inductor coil; and the first electrode and the second electrode are formed along the inductor coil.

In certain embodiments, the first electrode and second electrode are configured to generate a bipolar electrical field that passes through myocardial cells of the heart of the patient when the first electrode and second electrode are positioned on the heart of the patient.

In some embodiments, the generator is configured to be implanted in the patient.

Advantages of the systems, devices, and methods described herein can include improved defibrillation through the use of targeted electroporation. In addition, the systems, devices, and methods described herein can reduce the pain associated with cardiac defibrillation. As a result, the systems, devices, and methods described herein can provide effective ventricular fibrillation, as well as effective atrial defibrillation with reduced pain compared to standard defibrillation. The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 depicts a cross-sectional view of a human heart.

FIG. 2 depicts an example process for performing cardiac defibrillation using electroporation.

FIGS. 3-11 depict multiple embodiments of example electroporation systems in a heart, in accordance with embodiments provided herein.

### DETAILED DESCRIPTION

This disclosure describes methods and materials for treating atrial and ventricular fibrillation. For example, this document describes methods and devices to deliver reverse electroporation to cardiac tissue to treat atrial and ventricular fibrillation.

Electroporation is a technique that uses rapid bursts of DC current to non-thermally introduce multiple nanopores with the cells' walls of surrounding tissue. The methods and devices provided herein can deliver targeted electroporation to myocardial cells in the heart to cause ventricular defibrillation and/or atrial defibrillation without causing ablation or thermal injury to the cardiac tissue. By generating a pulsed electrical field and transmitting pulsed electrical vectors directly through the myocardial tissue without passing through other tissue of the patient (e.g., surrounding skeletal muscle or nerves), pain experienced by the patient during defibrillation via electroporation is greatly reduced compared to pain experienced during traditional defibrillation.

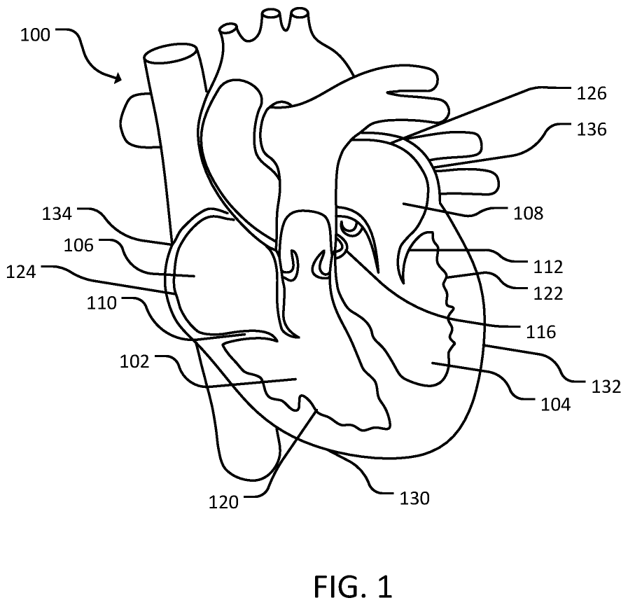

Referring to FIG. 1, a heart 100 includes a right ventricle 102, a left ventricle 104, a right atrium 106, and a left atrium 108. A tricuspid valve 110 is located between right atrium 106 and right ventricle 102. A mitral valve 112 is located between left atrium 108 and left ventricle 104. A semilunar valve 116 is located between left ventricle 104 and the aorta.

Fibrillation can occur in the right ventricle 102, the left ventricle 104, the right atrium 106, and/or the left atrium 108, and can result in cardiac arrest. As described in further detail below, devices and methods for administering electroporation to the right ventricle 102, the left ventricle 104, the right atrium 106, or the left atrium 108 to terminate fibrillation are provided herein. Using such devices and techniques, treatment of ventricular defibrillation or atrial defibrillation can be achieved while avoiding ablation or thermal injury to cardiac tissue.

Each of the right ventricle 102, the left ventricle 104, the right atrium 106, and the left atrium 108 includes an endocardial surface 120, 122, 124, 126 and an epicardial surface 130, 132, 134, 136, respectively. As described in further detail herein, electrodes may be positioned on the endocardial surface 120, 122, 124, 126, the epicardial surface 130, 132, 134, 136, or both, of one or more of the ventricles 102, 104 and atriums 106, 108 of the patient in order to perform reversible electroporation of myocardial cells of the respective ventricles 102, 104 and atriums 106, 108.

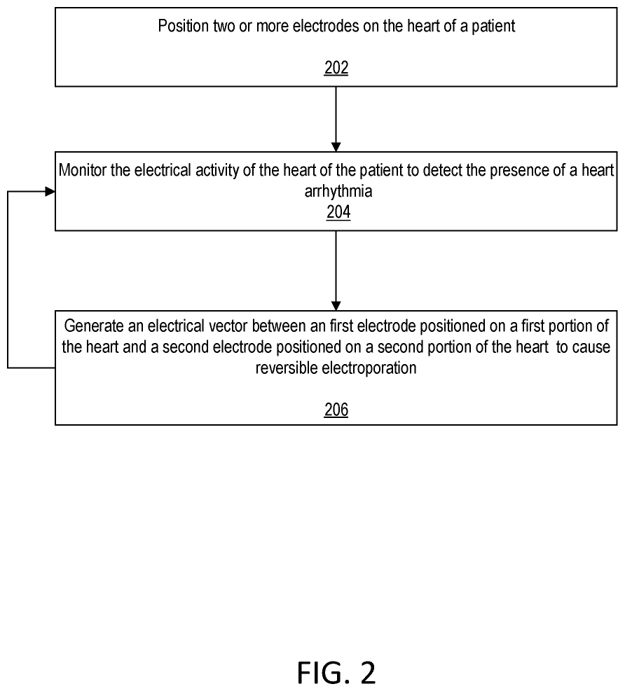

Referring to FIGS. 1, 2, and 11, a method for using reversible electroporation to conduct cardiac defibrillation will now be described.

Two or more electrodes 1102, 1104 are positioned on one or more surfaces of the heart 100 of a patient 10 (206). In some implementations, the electrodes 1102, 1104 include at least one cathode and at least one anode, which together generate a bipolar direct current electrical field when energy is supplied to the electrodes 1102, 1104. In some implementations, the electrodes 1102, 1104 are configured to generate a multi-polar direct current field. In some implementations, the electrodes 1102, 1104 are configured to generate a unipolar direct current field. In some cases, the polarity of each of the electrodes 1102, 1104 can be modified in order to generate a variety of different electrical fields and shocking vectors.

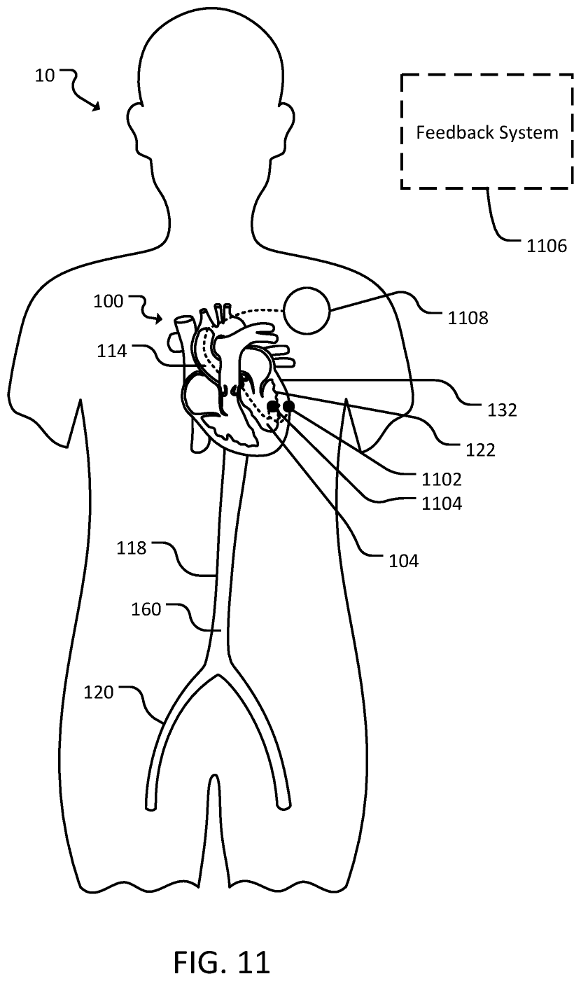

To perform ventricular defibrillation using reversible electroporation, the electrodes 1102, 1104 are placed on one or more surfaces of the one or both of ventricles 102, 104 of the patient, as depicted in FIG. 11. In some implementations, each of the electrodes 1102, 1104 is placed on the endocardial surface 120, 122 of a ventricle 102, 104. In some implementations, each of the electrodes 1102, 1104 is placed on an epicardial surface 130, 132 of a ventricle 102, 104. As can be seen in FIG. 11, in some implementations, one or more of the electrodes 1102, 1104 are placed on the endocardial surface 120, 122 of a ventricle 104 and one or more electrodes 1102, 1104 are placed on the epicardial surface 130, 132 of the ventricle 104. For example, an anode can be placed on the endocardial surface 120, 122 of the ventricle 102, 104 and a cathode can be placed on the epicardial surface 130, 132 of the ventricle 102, 104, or vice versa. In some implementations, electrodes 1102, 1104 are placed on the epicardial surface 130, 132 and/or the endocardial surface 120, 122 of both of the ventricles 102, 104.

Similarly, in order to perform atrial defibrillation via reversible electroporation, electrodes 1102, 1104 are placed on one or more surfaces of the atria 106, 108 of the patient. For example, in some implementations, each of the electrodes 1102, 1104 is placed on the endocardial surface 124, 126 of an atrium 106, 108. In some embodiments, each of the electrodes 1102, 1104 is placed on the epicardial surface 134, 136 of an atrium 106, 108. In some embodiments, one or more of the electrodes 1102, 1104 are placed on the endocardial surface 124, 126 of an atrium 106, 108 and one or more electrodes 1102, 1104 are placed on the epicardial surface 134, 136 of the same atrium 106, 108. For example, an anode can be placed on the endocardial surface 124, 126 of an atrium 106, 108 and a cathode can be placed on the epicardial surface 134, 136 of the same atrium 106, 108, or vice versa. In some implementations, electrodes 1102, 1104 are placed on the epicardial surface 134, 136 and/or the endocardial surface 124, 126 of both of the atriums 106, 108.

In some implementations, the electrodes 1102, 1104 are positioned on surfaces of the heart 100 that are proximate areas of the heart 100 with an increased likelihood of serving as a trigger for fibrillation. By positioning the electrodes 1102, 1104 to target particular regions of the heart 100, the electroporation caused by the electrodes can be targeted to areas of the heart 100 experiencing fibrillation, without interfering with conduction of other regions of the heart 100. For example, for atrial defibrillation, electrodes 1102, 1104 can be placed in the pericardial sinuses of the heart 100 in order to cause electroporation of the myocardial tissue surrounding the atrial cardiac nervous system (which is an area of tissue likely to cause atrial fibrillation), without affecting ventricular conduction.

Once the electrodes 1102, 1104 are positioned on one or more surfaces of the heart 100 of the patient 10, the electrical activity of the heart 100 of the patient 10 is monitored (204). For example, one or more of the electrodes 1102, 1104 can detect and record electrical signals generated by the heart 100. The electrodes 1102, 1104 can be communicably coupled to a feedback system 1106 to transmit electrical signals generated by the heart 100 and detected by the electrodes 1102, 1104 to the feedback system 1106. As discussed in further detail herein, the feedback system 1106 can modify the energy delivered to the electrodes 1102, 1104 in order to perform electroporation of the myocardial tissue proximate the electrodes 1102, 1104 in response to detecting fibrillation. In some cases, the electrical activity monitored by the electrodes 1102, 1104 can be displayed in real time and provide feedback regarding potential or ongoing atrial or ventricular fibrillation.

In some implementations, the electrodes 1102, 1104 can be used to determine a particular location within the heart 100 that fibrillation is occurring (e.g., electrodes 1102, 1104 can act as sensing electrodes). In response to identifying the location of the fibrillation, the feedback system 1106 can modify the energy delivered to one or more particular electrodes 1102, 1104 positioned closest to the location of fibrillation in order to generate bipolar electroporation shocking vectors targeting the myocardial tissue at the location of the fibrillation. In addition, the electrodes 1102, 1104 can be used to determine pre-fibrillation electrical abnormalities, and in response to detecting the abnormalities, the electrodes 1102, 1104 can be controlled such that energy is delivered to one or more particular electrodes 1102, 1104 positioned closest to the location of the abnormality in order to prevent cardiac fibrillation.

By placing multiple electrodes on various portions of the heart 100, different distinctive fields of energy delivery can be generated and cardiac abnormalities that are localized to particular regions of the heart 100 may be selectively treated with electroporation using one or more electrodes positioned proximate to the region of the heart 100 experiencing abnormal electrical activity. For example, in response to receiving signals from one or more electrodes indicating that a particular region of the heart 100 is experiencing abnormal activity, such as an abnormal change in heart rate, ventricular ectopy, or abnormal changes in amplitude, frequency or slew in the signals generated by one or more of the electrodes 1102, 1104, one or more electrodes 1102, 1104 proximate to the region of the heart 100 experiencing the abnormal electrical activity can be automatically (for example, via feedback system 1106) or manually (for example, via user input) controlled to provide electroporation sequences to the localized portion of the heart 100 experiencing abnormal electrical activity in order to defibrillate the region and/or prevent fibrillation.

A variety of techniques can be used by the electrodes 1102, 1104 to monitor electrical activity of the heart 100 and determine a particular location within the heart 100 that fibrillation is occurring. For example, various algorithms can be used to analyze and interpret the electrical signals measured by the electrodes 1102, 1104, and based on monitoring the electrical signals across various portions of the heart of a patient, voltage gradients, rapid heart rates, unstable rhythms (e.g., changes in cardiac cycle length), repolarization gradients, early unanticipated depolarizations, etc. can be detected, which can indicate the presence of cardiac fibrillation. As will be described in further detail herein, based on detecting the presence and location of potential cardiac fibrillation, targeted electroporation can be delivered by the electrodes 1102, 1104 to the portions of cardiac tissue detected by the electrodes 1102, 1104 as initiating and/or undergoing fibrillation.

For example, in some implementations, the electrodes 1102, 1104 can function as rate counter sensors that detect elevated rates of electrical activity across portion of the heart 100, which can indicate the presence of fibrillation. In some implementations, in response to the electrodes 1102, 114 detecting higher rates of electrical activity in a particular area of the heart 100, electroporation can be provided by one or more electrodes 1102, 1104 proximate the area of tissue that was first detected as having higher rates of electrical activity, as will be described in further detail herein. In addition, in some implementations, if after providing targeted electroporation, the electrodes 1102, 1104 detect that increased levels of electrical activity are still present, electroporation will be provided by all of the electrodes 1102, 1104.

In some implementations, the electrodes 1102, 1104 are configured to detect cardiac fibrillation by measuring and monitoring voltages gradients across portions of the heart 100. For example, if the electrodes 1102, 1104 detect that an abrupt change in the voltage gradient across a portion of the heart 100 (e.g., across a ventricle 102, 104 or an atrium 106, 108), the electrodes 1102, 1104 can deliver targeted electroporation to the portion of the heart 100 detected as experiencing the sudden change in voltage gradient in order to terminate fibrillation, as will be described in further detail herein. Further, in some implementations, the electrodes 1102, 1104 can be configured to detect regions of the heart 100 experiencing delayed or early repolarization (repolarization gradients), and areas of high repolarization gradient within a relatively limited space (e.g., the distance between electrodes measuring the gradient) will be targeted for electroporation delivery.

In response to detecting a cardiac arrhythmia (e.g., atrial or ventricular fibrillation), electricity is provided to the two or more electrodes 1102, 1104 positioned on the heart 100 of the patient 10 in order to generate a bipolar electrical field between the electrodes 1102, 1104. For example, as depicted in FIG. 11, each of the electrodes 1102, 1104 can be electrically coupled to a generator 1108, which provides energy to the electrodes 1102, 1104. In some implementations, the generator 1108 provides pulsating direct current to the electrodes 1102, 1104. In some implementations, the generator 1108 is communicably coupled to the feedback system 1106, and provides energy to one or more of the electrodes 1102, 1104 in response to a signal received from the feedback system 1106 indicating the presence and/or location of fibrillation within the heart 100.

As depicted in FIG. 11, the generator 1108 can be implanted in the patient 10, for example, under the skin of the patient 10 near the clavicle bone of the patient 10. In some implementations, the generator 1108 can be placed in the mediastinum of the patient 10. The generator 1108 can be a standard, implantable generator, such as the type of generator used in conventional defibrillators. In some implementations, the generator 1108 is a specialized generator configured to generate customizable waveforms for providing electroporation via electrodes 1102, 1104. For example, the generator 1108 can be configured to deliver electricity to the electrodes 1102, 1104 in varying pulse widths from nanosecond pulse widths to multi-second pulse widths. In addition, the generator 1108 can be configured to provide a range of voltages of electricity to the electrodes 1102, 1104 from nanovolts to several kilovolts. The generator 1108 can be configured to generate a variety of waveforms, including, but not limited to monophasic, biphasic, quadraphasic, and multiphasic waveforms. In addition, the generator 1108 may control the delivery sequence of electricity provided to the electrodes 1102, 1104, such as providing bursts of electricity, ramped delivery (e.g., based on detecting shortened cardiac cycle lengths), and/or sequences of one or more of the above-described waveforms. The delivery of electricity to the electrodes 1102, 1104 by the generator 1108 can be controlled based on applying unique algorithms to the electrical signals received from the electrodes 1102, 1104.

In some cases, electrodes 1102, 1104 can provide pulses of direct current in order to generate a pulsed electrical field between the electrodes 1102, 1104. In some implementations, the electrodes 1102, 1104 can provide electrical pulses with a pulse width in a range of about 1 nanosecond to about 300 microseconds and generate an electrical field with an electrical potential of about 0.2 microCoulombs to about 30 milliCoulombs. In some implementations, the electrodes 1102, 1104 can provide electrical pulses with a pulse width in a range of about 10 microseconds to about 100 microseconds and generate an electrical field with an electrical potential of about 2 microCoulombs to about 10 microCoulombs. In some cases, the pulses can have a delay (e.g., 1-2 seconds between pulses). In some cases, the pulses can be delivered at range of 200 volts to 10,000 volts.

The particular electrical delivery sequence used for conducting reversible electroporation may vary from patient to patient. However, in general, nanosecond scale pulse widths combined with up to a thousand volts amplitude pulses can be used to provide reversible electroporation without thermal damage. In addition, microsecond to second pulse widths combined with microvolt or millivolt amplitude pulses can be used to provide reversible electroporation without thermal damage.

In some implementations, electrodes 1102, 1104 are configured to provide a variety of sequences of pulses of direct current, such that the electrical pulses provided by the electrodes 1102, 1104 can be adjusted according to parameters that are specific to the particular patient 10 receiving the electroporation treatment. For example, in one implementation, electrodes 1102, 1104 can be configured to provide electrical pulses with a pulse width of about 200 nanoseconds and deliver 10,000 volts of electrical energy in a sequence of 200 pulses delivered at a range of 1-15 Hz. In one example, electrodes 1102, 1104 can be configured to provide electrical pulses with a pulse width of about 100 nanoseconds and deliver 5,000 volts of electrical energy in a sequence of 100 pulses delivered at 6 Hz.

In some implementations, a polarity can be individually selected for each electrode 1104. Further, in some implementations, one or more of the electrodes 1102, 1104 can be selectively disabled. For example, energy may be provided only to a subset of the electrodes 1102, 1104 positioned on the heart 100 in order to target a particular region of the heart 100.

By delivering electrical pulses in the ranges described above to the myocardial tissue, reversible electroporation of the myocardial tissue is performed.

Electroporation of the myocardial tissue using electrodes 1102, 1104 results in temporary poration of the cell membrane of the myocardial cells within the pulsed electrical field generated by the electrodes 1102, 1104. The temporary poration of the cell membrane of myocardial cells causes a rush of ions into the cardiac ion channels of the porated myocardial cells, which temporarily affects cardiac ion channel conduction within the porated cells. This temporary change in cardiac ion channel conduction temporarily paralyzes the porated myocardial cells, which serves to terminate fibrillation in the respective region of the heart 100.

In some implementations, the electrodes 1102, 1104 are positioned on the heart 100 such that the pulsed electrical field generated by the electrodes 1102, 1104 passes through a critical mass of myocardial tissue that is large enough to ensure termination of a cardiac arrhythmia. For example, in systems for performing ventricular defibrillation, the entire ventricular mass may be within the field of the electrodes 1102, 1104. However, based on certain electrical activity detected by the electrodes 1102, 1104, such as the rate of local electrical signals detected, the regularity of local electrical signals detected, the change in voltage gradients, absolute values of voltage gradients, and repolarization gradients, the electroporation field and energy delivery provided by the electrodes 1102, 1104 may be targeted to portions of the ventricular mass in which the irregular signals were detected. By targeting the electroporation field and energy delivery to specific portions of cardiac tissue in which irregular electrical activity is detected, the electroporation provided by the electrodes 1102, 1104 may be delivered to portions of the heart 100 constituting less than 10% of the total myocardial mass, while still successfully terminating ventricular fibrillation.

As previously discussed, by delivering the electrical vectors of the pulsating electrical field directly through the myocardial tissue that is experiencing fibrillation, without passing electricity through other surrounding tissue of the patient 10 (e.g., surrounding skeletal muscle or nerves), pain experienced by the patient during defibrillation via electroporation is greatly reduced compared to pain experienced during traditional defibrillation.

In some implementations, after delivering a first electrical shocking vector, the electrodes 1102, 1104 continue to monitor the electrical activity of the heart 100 to determine whether the initial electroporation was effective in terminating the fibrillation. If, based on the electrical signals generated by the heart 100 and detected by one or more of the electrodes 1102, 1104, it is determined that fibrillation is still present, energy can be provided to the electrodes 1102, 1104 to generate additional pulsating electrical vectors. In some implementations, the subsequent electrical vectors have different magnitude and/or direction from the initial electrical vector in order to target different myocardial tissue. In some implementations, these alternate vectors are applied by the electrodes 1102, 1104 in a rapid sequence until it is detected that the fibrillation is terminated.

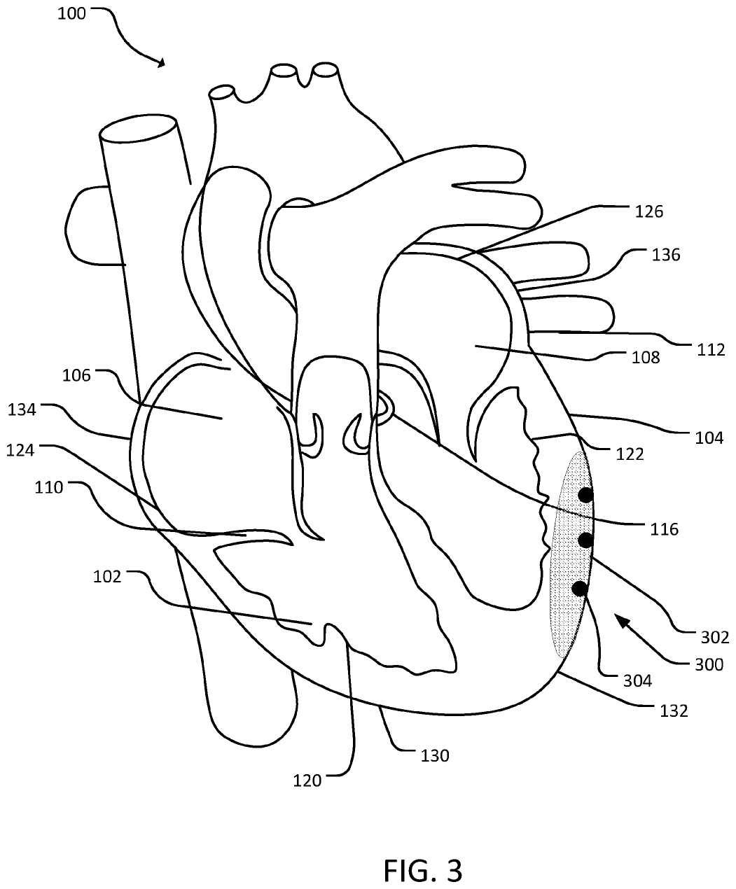

FIGS. 3-10 depict example systems for positioning electrodes on one or more surfaces of the heart 100 in order to perform cardiac defibrillation using electroporation, as described above.

For example, FIGS. 3-5 depict example systems for positioning electrodes on one or more surfaces of a ventricle in order to perform ventricular defibrillation using reversible electroporation. Referring to FIG. 3, a first example system 300 includes a mesh (or lattice) 302. A set of electrodes 304 are attached to the mesh 302. The mesh 302 can be made of one or more biocompatible materials to allow for long-term implantation of the system 300 in the patient 10. For example, the mesh 302 can be made of dielectric or semiconductor materials, including, but not limited to, graphite, platinum, and silicone. In some implementations, the mesh 302 can be made of a conductive material (e.g., copper) coated in an insulating material (e.g., nitinol), with portions of the insulated coating removed to expose the underlying conductive material, such that the exposed conductive material functions as a series of electrodes.

As depicted in FIG. 3, the mesh 302 can be attached to epicardial surface 132 of the left ventricle 104. In some implementations, the mesh 302 is attached to the epicardial surface 132 via the transverse sinus of the pericardial space using sutures to and/or ligatures coupled to the mesh and placed around the great arteries or the left atrial appendage. In some implementations, the mesh 302 is attached to the parietal pericardium of the heart 100 proximate the left ventricle 104 at areas distal from the phrenic nerve. In some implementations, the electrodes 304 are anchored into the myocardium or into the parietal pericardium upon attachment of the mesh 302 to the epicardial surface 132 using an attachment device, such as with an active helix.

When the mesh 302 is attached to epicardial surface 132 of the left ventricle 104, the electrodes 304 are positioned against the epicardial surface 132 of the left ventricle 104 in contact with myocardial cells of the left ventricle 104. In some implementations, at least one of the electrodes 304 is an anode and at least one of the electrodes 304 is a cathode. As such, when energy is supplied to the electrodes 304, a bipolar electrical field is generated and the electrical pulses generated by the electrodes 304 are transmitted to the myocardial cells forming the epicardial surface 132 of the left ventricle 104.

While FIG. 3 depicts the system 300 being attached to the left ventricle 104, in some embodiments, the system 300 is attached to the right ventricle 102. For example, mesh 302 can be attached to the epicardial surface 130 of the right ventricle 102 to position the electrodes 304 against the epicardial surface 130 of the right ventricle 102 in contact with myocardial cells of the right ventricle 102. In such an arrangement, electrical pulses generated by the electrodes 304 are transmitted to the myocardial cells forming the epicardial surface 130 of the right ventricle 102.

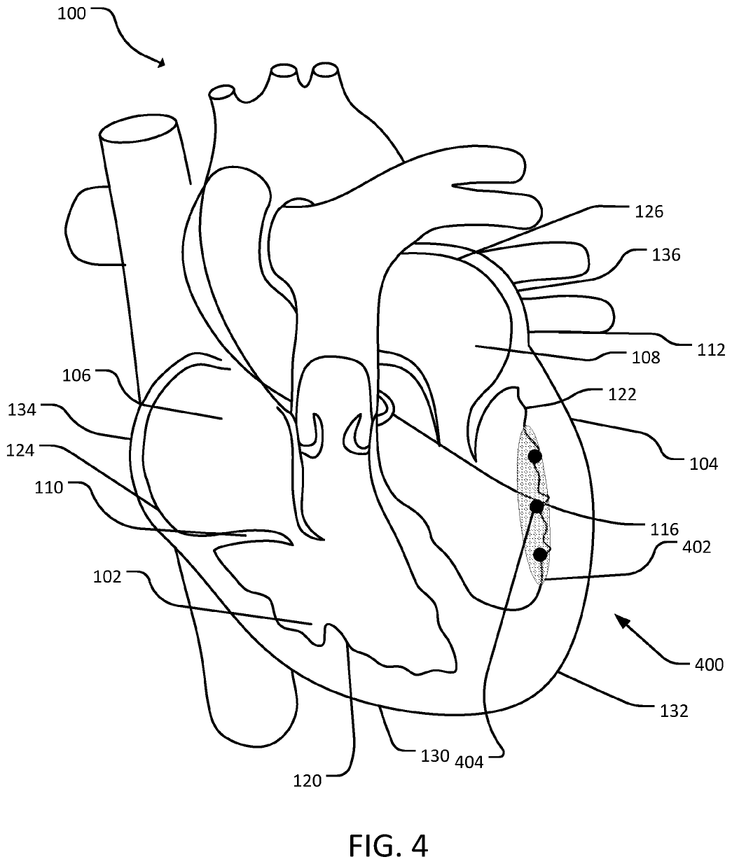

FIG. 4 depicts another example system 400 for positioning electrodes on a surface of a ventricle in order to perform ventricular defibrillation using reversible electroporation. Example system 400 includes a mesh (or lattice) 402 with electrodes 404 attached to the mesh 402. The mesh 402 can be made of one or more biocompatible materials to allow for long-term implantation of the system 400 in the patient. For example, the mesh 402 can be made of dielectric or semiconductor materials, including, but not limited to, graphite, platinum, and silicone. In some implementations, the mesh 402 can be made of a conductive material (e.g., copper) coated in an insulating material (e.g., nitinol), with portions of the insulated coating removed to expose the underlying conductive material, such that the exposed conductive material can function as a series of electrodes.

As depicted in FIG. 4, the mesh 402 can be attached to endocardial surface 122 of the left ventricle 104. In some implementations, the mesh 402 is attached to the endocardial surface 122 using one or more attachment devices, including, but not limited to, active helices, tines, and expandable elements configured to engage the true apex, endocavitary structures, or trabeculations endocardially. When the mesh 402 is attached to endocardial surface 122 of the left ventricle 104, the electrodes 404 are positioned against the endocardial surface 122 of the left ventricle 104 in contact with myocardial cells of the left ventricle 104. In some implementations, at least one of the electrodes 404 is an anode and at least one of the electrodes 404 is a cathode. As such, when electricity is supplied to the electrodes 404, a bipolar electrical field is generated and the electrical pulses generated by the electrodes 404 are transmitted to the myocardial cells forming the endocardial surface 122 of the left ventricle 104.

While FIG. 4 depicts the system being attached to the left ventricle 104, in some embodiments, the system 400 is attached to the right ventricle 102. For example, mesh 402 can be attached to endocardial surface 120 of the right ventricle 102 to position the electrodes 404 against the endocardial surface 120 of the right ventricle 102 in contact with myocardial cells of the right ventricle 102. In such an arrangement, electrical pulses generated by the electrodes 404 are transmitted to the myocardial cells forming the endocardial surface 120 of the right ventricle 102.

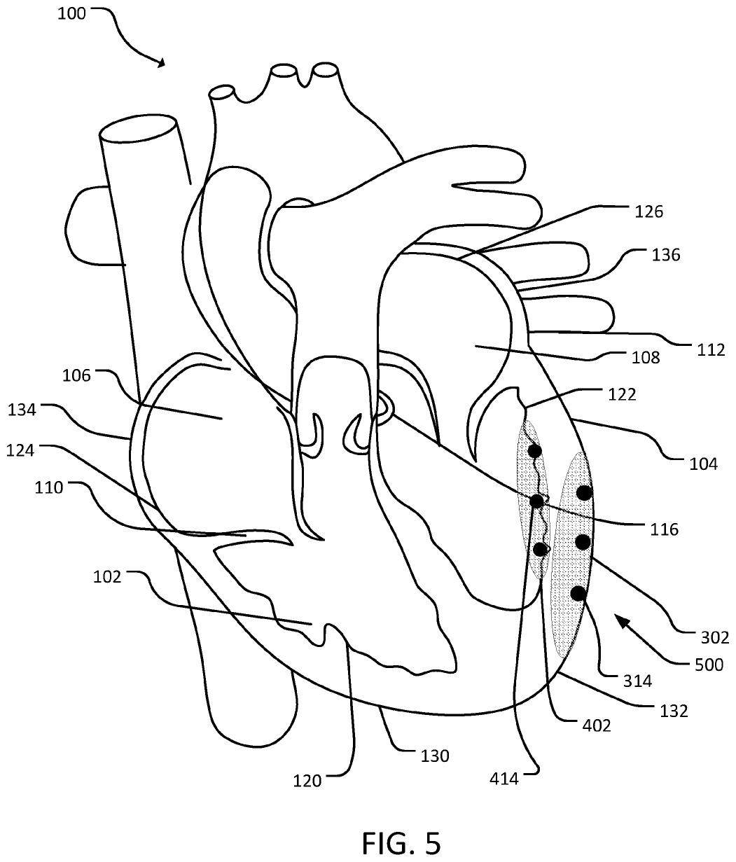

FIG. 5 depicts an example system 500 for positioning electrodes on multiple surfaces of a ventricle in order to perform ventricular defibrillation using reversible electroporation. Example system 500 includes a first mesh (or lattice) 302 with a first electrodes 314 attached to the first mesh 302 and a second mesh (or lattice) 402 with a second set of electrodes 414 attached to the second mesh 402. As depicted in FIG. 5, the first mesh 302 is attached to epicardial surface 132 of the left ventricle 104 and the second mesh 402 is attached to endocardial surface 122 of the left ventricle 104. As such, the first set of electrodes 314 are positioned against the epicardial surface 132 of the left ventricle 104 and the second set of electrodes 414 are positioned against the endocardial surface 122 of the left ventricle 104. In some implementation, the first set of electrodes 314 are cathodes and the second set of electrodes are anodes 414 and when energy is provided to the sets of electrodes 314, 414 (e.g., from generator 1108) a bipolar electrical field is generated between the sets of electrodes 314, 414 through the wall of the left ventricle 104 and the electrical pulses generated by the electrodes 314, 414 are transmitted to the myocardial cells forming the wall of the left ventricle 104. Alternatively, the first set of electrodes 314 can be anodes and the second set of electrodes can be cathodes 414. In some embodiments, each set of electrodes 314, 414 of the system 500 includes both anodes and cathodes. In some implementations, intraseptal or transmyocardial anchoring of the electrodes 314, 414 is performed using a clamshell-type device so as to position the electrodes 314, 414 on either side of the intraventricular septum, or to position electrodes 314, 414 on both the endocardial surface 122 and the epicardial surface 132.

While FIG. 5 depicts the system 500 being attached to the left ventricle 104, in some embodiments, the system 500 is attached to the right ventricle 102. For example, the first mesh 302 can be attached to epicardial surface 130 of the right ventricle 102 and the second mesh 402 can attached to endocardial surface 120 of the right ventricle 102. As such, the first set of electrodes 314 can be positioned against the epicardial surface 130 of the right ventricle 102 and the second set of electrodes 414 can positioned against the endocardial surface 120 of the right ventricle 102 to generate a bipolar electrical field that passes through the wall of the right ventricle 102.

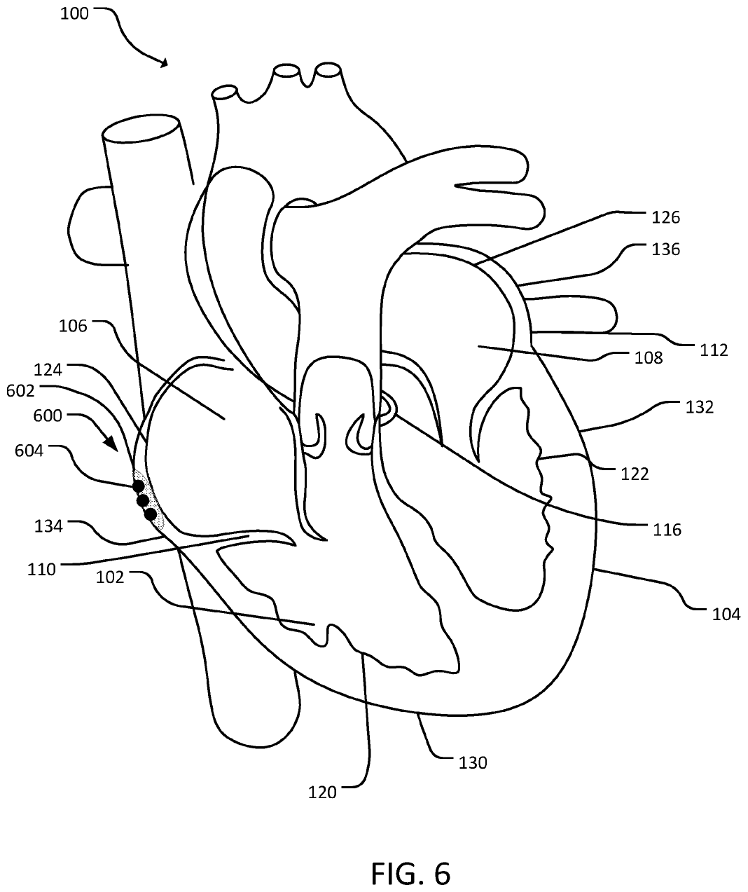

FIGS. 6-8 depict example systems for positioning electrodes on one or more surfaces of an atrium in order to perform atrial defibrillation using reversible electroporation. Referring to FIG. 6, a first example system 600 includes a mesh (or lattice) 602 with electrodes 604 attached to the mesh 602. The mesh 602 can be made of one or more biocompatible materials to allow for long-term implantation of the system 600 in the patient. For example, the mesh 602 can be made of dielectric or semiconductor materials, including, but not limited to, graphite, platinum, and silicone. In some implementations, the mesh 602 can be made of a conductive material (e.g., copper) coated in an insulating material (e.g., nitinol), with portions of the insulated coating removed to expose the underlying conductive material, such that the exposed conductive material can function as a series of electrodes.

As depicted in FIG. 6, the mesh 602 can be attached to epicardial surface 134 of the right atrium 106. In some implementations, the mesh 602 is attached to the epicardial surface 134 via the transverse sinus of the pericardial space using sutures and/or ligatures coupled to the mesh and placed around the great arteries or the appendage. In some implementations, the mesh 602 is attached to the parietal pericardium of the heart 100 proximate the right atrium 106 at areas distal from the phrenic nerve. In some implementations, the electrodes 604 are anchored into the myocardium or into the parietal pericardium upon attachment of the mesh 602 to the epicardial surface 134 using an attachment device, such as with an active helix.

When the mesh 602 is attached to epicardial surface 134 of the right atrium 106, the electrodes 604 are positioned against the epicardial surface 134 of the right atrium 106 in contact with myocardial cells of the right atrium 106. In some implementations, at least one of the electrodes 604 is an anode and at least one of the electrodes 604 is a cathode. As such, when energy is supplied to the electrodes 604, a bipolar electrical field is generated and the electrical pulses generated by the electrodes 604 are transmitted to the myocardial cells forming the epicardial surface 134 of the right atrium 106.

While FIG. 6 depicts the system being attached to the right atrium 106, in some embodiments, the system 300 is attached to the left atrium 108. For example, mesh 602 can be attached to epicardial surface 136 of the left atrium 108 to position the electrodes 604 against the epicardial surface 136 of the left atrium 108 in contact with myocardial cells of the left atrium 108. In such an arrangement, electrical pulses generated by the electrodes 604 are transmitted to the myocardial cells forming the epicardial surface 136 of the left atrium 108.

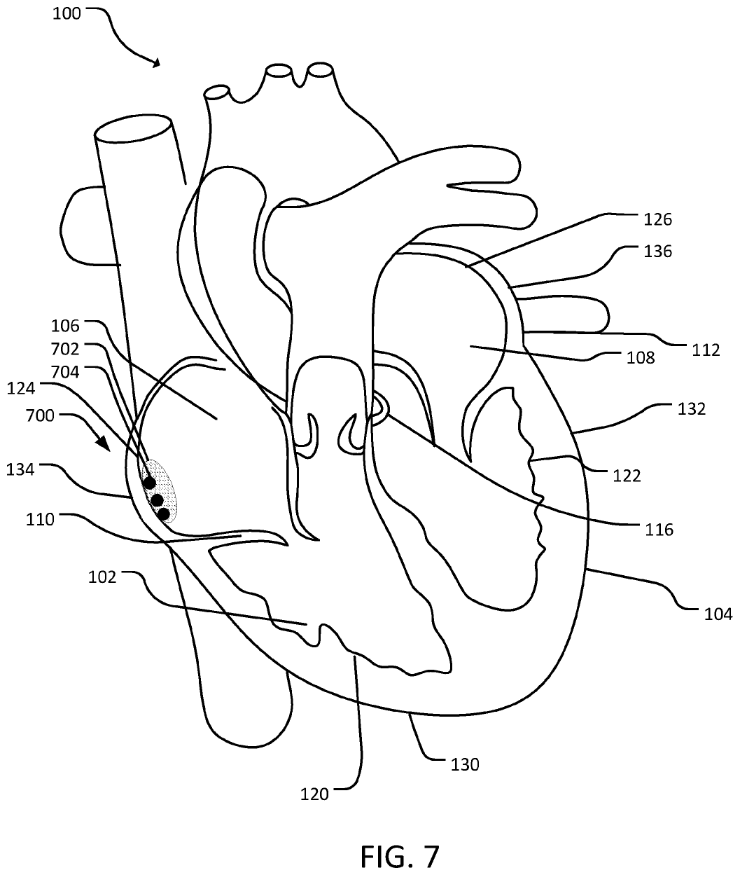

FIG. 7 depicts another example system 700 for positioning electrodes on a surface of an atrium. Example system 700 includes a mesh (or lattice) 702 with electrodes 704 attached to the mesh 702. The mesh 702 can be made of one or more biocompatible materials to allow for long-term implantation of the system 700 in the patient. For example, the mesh 702 can be made of dielectric or semiconductor materials, including, but not limited to, graphite, platinum, and silicone. In some implementations, the mesh 702 can be made of a conductive material (e.g., copper) coated in an insulating material (e.g., nitinol), with portions of the insulated coating removed to expose the underlying conductive material, such that the exposed conductive material can function as a series of electrodes.

As depicted in FIG. 7, the mesh 702 can be attached to endocardial surface 124 of the right atrium 106. In some implementations, the mesh 702 is attached to the endocardial surface 124 using one or more attachment devices, including, but not limited to, active helices, tines, and expandable elements configured to engage the true apex, endocavitary structures, or trabeculations endocardially. When the mesh 702 is attached to endocardial surface 124 of the right atrium 106, the electrodes 704 are positioned against the endocardial surface 124 of the right atrium 106 in contact with myocardial cells of the right atrium 106. At least one of the electrodes 704 is an anode and at least one of the electrodes 704 is a cathode. As such, when electricity is supplied to the electrodes 704, a bipolar electrical field is generated and the electrical pulses generated by the electrodes 704 are transmitted to the myocardial cells forming the endocardial surface 124 of the right atrium 106.

While FIG. 7 depicts the system 700 being attached to the right atrium 106, in some embodiments, the system 700 is attached to the left atrium 108. For example, mesh 702 can be attached to endocardial surface 126 of the left atrium 108 to position the electrodes 704 against the endocardial surface 126 of the left atrium 108 in contact with myocardial cells of the left atrium 108. In such an arrangement, electrical pulses generated by the electrodes 704 are transmitted to the myocardial cells forming the endocardial surface 126 of the left atrium 108.

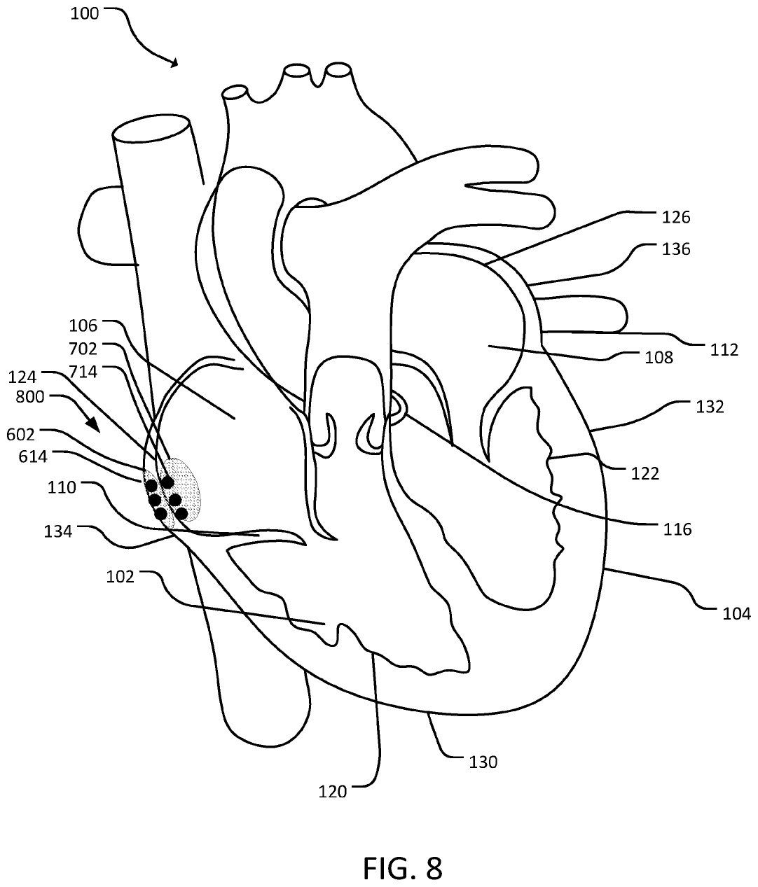

FIG. 8 depicts an example system 800 for positioning electrodes on multiple surfaces of an atrium in order to perform ventricular defibrillation using reversible electroporation. Example system 800 includes a first mesh (or lattice) 602 with a first electrodes 614 attached to the first mesh 602 and a second mesh (or lattice) 702 with a second set of electrodes 714 attached to the second mesh 702. As depicted in FIG. 8, the first mesh 602 is attached to epicardial surface 134 of the right atrium 106 and the second mesh 702 is attached to endocardial surface 124 of the right atrium 106. As such, the first set of electrodes 614 is positioned against the epicardial surface 134 of the right atrium 106 and the second set of electrodes 714 is positioned against the endocardial surface 124 of the right atrium 106. In some implementations, the first set of electrodes 614 is a set of cathodes and the second set of electrodes 714 is a set of anodes, and, as a result, a bipolar electrical field is generated between the sets of electrodes 614, 714 through the wall of the right atrium 106 when energy is provided to the sets of electrodes 614, 714. Alternatively, in some implementations, the first set of electrodes 614 is a set of anodes and the second set of electrodes 714 is a set of cathodes. In some embodiments, each of the sets of electrodes 614, 714 of the system 800 includes both anodes and cathodes. In some implementations, intraseptal or transmyocardial anchoring of the electrodes 614, 714 is performed using a clamshell-type device so as to position the electrodes 614, 714 on either side of the intraventricular septum, or top position electrodes 614, 714 on both the endocardial surface 124 and the epicardial surface 134.

While FIG. 8 depicts the system 800 being attached to the right atrium 106, in some embodiments, the system 800 is attached to the left atrium 108. For example, the first mesh 602 can be attached to epicardial surface 136 of the left atrium 108 and the second mesh 702 can attached to endocardial surface 126 of the left atrium 108. As such, the first set of electrodes 614 can be positioned against the epicardial surface 136 of the left atrium 108 and the second set of electrodes 718 can positioned against the endocardial surface 126 of the left atrium 108 to generate a bipolar electrical field that passes through the wall of the right ventricle 102.

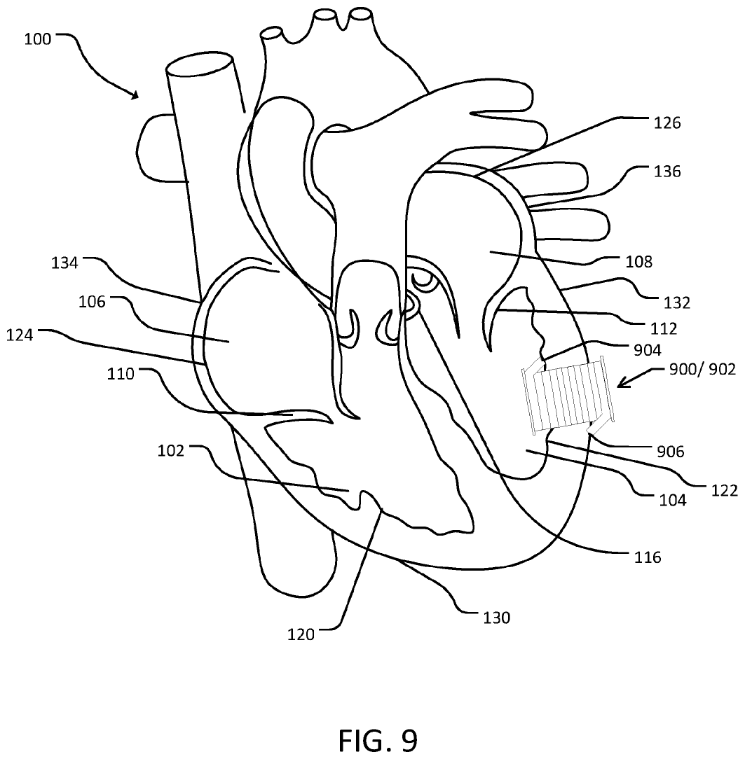

FIG. 9 depicts another example system 900 for positioning electrodes on one or more surfaces of a ventricle of the heart 100. As depicted in FIG. 9, the system 900 includes an inductor coil 902. The inductor coil 902 includes two exposed ends 904, 906 that function as electrodes to perform electroporation of myocardial cells. As depicted in FIG. 9, the inductor coil 902 can be inserted through the wall of the left ventricle 104 such that a first exposed end 904 of the inductor coil 902 contacts the endocardial surface 122 of the left ventricle 104 and a second exposed end 906 contacts the epicardial surface 132 of the left ventricle 104. Based on this arrangement, when energy is provided to the inductor coil 902 (e.g., from generator 1108), a bipolar electrical field is generated by the inductor coil 902 and transmitted through the wall of the left ventricle 104 between the ends 904, 906 of the inductor coil 902. In some embodiments, the inductor coil 902 is a partially insulated coil. In some embodiments, the inductor coil 902 is a segmented coil.

In some implementations, the inductor coil for positioning electrodes on one or more surfaces of a ventricle 102, 104 of the heart 100 may be contained within another outer coil, such that the device contains concentric coils that together function together as an anode and cathode. In some implementations, the inductor coil for positioning electrodes on one or more surfaces of a ventricle 102, 104 of the heart 100 is made of a conducting element covered in an insulating material that defines one or more discontinuities, which expose underlying portions of the conducting material, and the exposed portions of conducting material function as a series of electrodes. For example, laser etching can be applied to portions of the insulating material to expose underlying portions of the conducting material, and the insulated space between the exposed portions of conducting material allows the exposed portions of conducting material to function as bipoles, multipoles, etc., for electrical sensing and electroporation delivery. In some implementations, standard defibrillator coils may be attached as one or more limbs of the electroporation delivery field and sequence, for example, when a standard defibrillator coil is already implanted in the patient 10 for a standard defibrillator.

While FIG. 9 depicts the inductor coil 902 passing through the wall of the left ventricle 104 and contacting both the endocardial surface 122 and the epicardial surface 132 of the left ventricle 104, in some embodiments, the inductor coil 902 is positioned on and contacts only the endocardial surface 122 of the ventricle 104. In some implementations, the inductor coil 902 is positioned on and contacts only the epicardial surface 132 of the ventricle 104. In some implementations, a first inductor coil is positioned on the endocardial surface 122 of the ventricle 104 and a second inductor coil is positioned on the epicardial surface 132 of the ventricle 104, and a pulsed electrical field is generated along and between the inductor coils.

In addition, while FIG. 9 depicts the system 900 being attached to the left ventricle 104, in some embodiments, the system 900 is attached to the right ventricle 102. For example, inductor coil 902 can be inserted through the wall of the right ventricle 102 such that a first exposed end 904 of the inductor coil 902 contacts the endocardial surface 120 of the right ventricle 102 and a second exposed end 906 contacts the epicardial surface 130 of the right ventricle 102. As such, a bipolar electrical field can be generated along the inductor coil 902 between the ends 904, 906 of the inductor coil 902 and passes through the wall of the right ventricle 102.

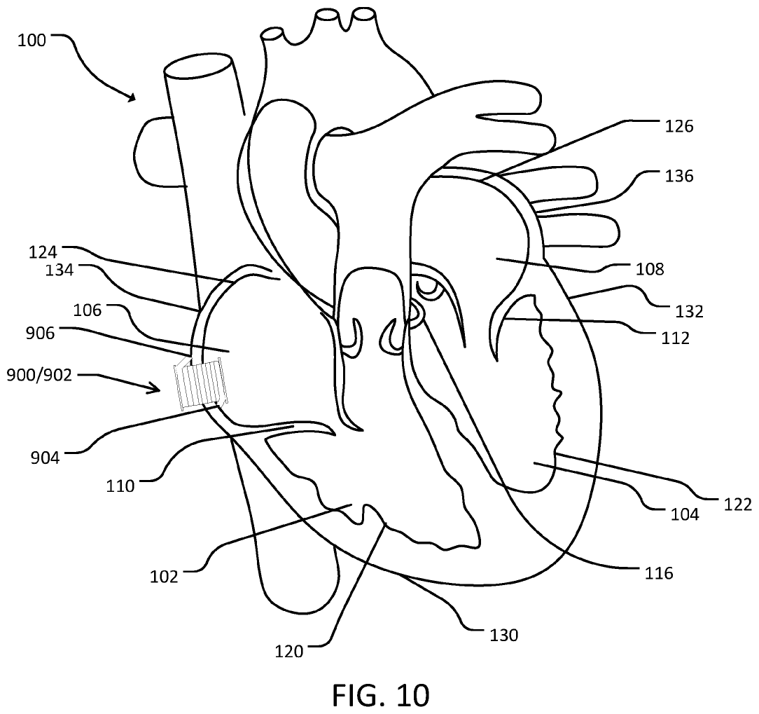

The inductor coil attachment system 900 can also be used for positioning electrodes on one or more surfaces of an atrium of the heart 100. For example, FIG. 10 depicts the inductor coil attachment system 900 coupled to the right atrium 106 of the heart. As depicted in FIG. 10, the inductor coil 902 can be inserted through the wall of the right atrium 106 such that a first exposed end 904 of the inductor coil 902 contacts the endocardial surface 124 of the atrium 106 and a second exposed end 906 contacts the epicardial surface 134 of the atrium 106. Based on this arrangement, when energy is provided to the inductor coil 902, a bipolar electrical field is generated along the inductor coil 902 between the ends 904, 906 of the inductor coil 902 and passes through the wall of the right atrium 106.

In some implementations, the inductor coil for positioning electrodes on one or more surfaces of an atrium 106, 108 of the heart 100 may be contained within another outer coil. In some implementations, the inductor coil for positioning electrodes on one or more surfaces of an atrium 106, 108 of the heart 100 is made of a conducting element covered in an insulating material that defines one or more discontinuities, which expose underlying portions of the conducting material, and the exposed portions of conducting material function as a series of electrodes. For example, laser etching can be applied to portions of the insulating material to expose underlying portions of the conducting material, and the insulated space between the exposed portions of conducting material allows the exposed portions of conducting material to function as bipoles, multipoles, etc., for electrical sensing and electroporation delivery. In some implementations, standard defibrillator coils may be attached as one or more limbs of the electroporation delivery field and sequence, for example, when a standard defibrillator coil is already implanted in the patient 10 for a standard defibrillator.

While FIG. 10 depicts the inductor coil 902 passing through the wall of the right atrium 106 and contacting both the endocardial surface 124 and the epicardial surface 134 of the right atrium 106, in some embodiments, the inductor coil 902 is positioned on and contacts only the endocardial surface 124 of the right atrium 106. In some implementations, the inductor coil 902 is positioned on and contacts only the epicardial surface 134 of the right atrium 106. In some implementations, a first inductor coil is positioned on the endocardial surface 124 of the right atrium 106 and a second inductor coil is positioned on the epicardial surface 134 of the right atrium 106, and a pulsed electrical field is generated along and between the inductor coils.

In addition, while FIG. 10 depicts the system 900 being attached to the right atrium 106, in some embodiments, the system 900 is attached to the left atrium 108. For example, inductor coil 902 can be inserted through the wall of the left atrium 108 such that a first exposed end 904 of the inductor coil 902 contacts the endocardial surface 126 of the left atrium 108 and a second exposed end 906 contacts the epicardial surface 136 of the left atrium 108. As such, a bipolar electrical field can be generated through the wall of the left atrium 108 between the ends 904, 906 of the inductor coil 902.

The inductor coil system 900 can also be used for positioning electrodes within the myocardium of the heart 100. For example, system 900 can include one or more intramyocardial inductor coils 902 that are positioned within the myocardium of one or more ventricles 102, 104 and/or atria 106, 108 of the heart 100 such that both of the exposed ends 904, 906 of the inductor coil 902 serving as electrodes are positioned within the myocardium. By utilizing an intramyocardial inductor coil 902 to position both electrodes 904, 906 of the coil 902 within the myocardium of the heart 100, ventricular or atrial fibrillation originating from myocardial tissue proximate the coil 902 can be terminated locally by delivering an electroporation field to the myocardial tissue using the inductor coil 902, which results in reduced pain to the patient compared to standard defibrillation shocks.

While certain embodiments have been described above, other embodiments are possible.

For example, while the electrodes of the electroporation defibrillation system have been described as being positioned on a surface of the heart, the electrodes can alternatively or additionally be placed within the cardiac venous system. For example, in some implementations, electrodes used for electroporation defibrillation are positioned within the venous system in each ventricle 102, 104 of the heart 100. In addition, one or more electrodes, including one or more inductor coils, can be positioned within the superior vena cava, coronary veins, or other veins or vessels. Electrodes (such as inductor coils) positioned within the cardiac vasculature can serve as return electrodes for any one of the electroporation systems described herein when the systems are implanted within the heart and vasculature.

In addition, while the electrodes of the electroporation defibrillation system have been described as being attached to the heart using a mesh, lattice, or electrical coil, other electrode attachment devices can be used. For example, in some implementations, the electrodes can be attached to rings of biocompatible material that are attached to one or more surfaces of the heart. In some implementations, one or more attachment devices, such as tines, active helixes, rings, curved hooks, clamshell-type devices, and clamps, can be used to anchor the electrodes to endocavitary structures, such as the papillary muscle, prominent trabeculations, or the supraventricular crest and equivalent structures.

Further, while the example systems depicted in FIGS. 3-11 each depict the use of a single type of electrode attachment device, any number and combination of electrode attachment devices can be used. For example, an electroporation system can include both mesh attachment devices (e.g., mesh 302) and inductor coils (e.g., inductor coil 902), and electrical fields can be generated between the electrodes on each of the attachment devices. In addition, while the example systems depicted in FIGS. 3-11 depict the use of 1 or 2 attachment devices, any suitable number of electrode attachment devices can be used.

While the example systems depicted in FIGS. 3-11 include 2 to 6 electrodes, other numbers of electrodes may be used to perform electroporation defibrillation. In addition, while FIGS. 3-11 depict the electrodes being placed on surfaces of either a single ventricle or a single atrium of the heart 100, electrodes may be placed on multiple surfaces of the heart, including on surfaces of one or more ventricles and one or more atriums. By placing multiple electrodes on various portions of the heart 100, cardiac abnormalities that are localized to particular regions of the heart may be selectively treated with electroporation using one or more electrodes positioned proximate to the region of the heart 100 experiencing abnormal electrical activity.

In addition, while FIGS. 9 and 10 each depict an electroporation system 900 that includes a single inductor coil 902, multiple inductor coils may be used to perform defibrillation. For example, one or more inductor coils 902 contained within the myocardium of one or more ventricles 102, 104 and/or atria 106, 108 of the heart 100 and one or more inductor coils 902 with exposed ends 904, 906 positioned on the epicardial and/or endocardial surfaces of one or more ventricles 102, 104 and/or atria 106, 108 may be utilized in order to improve localized defibrillation. For example, the electroporation system 900 can include multiple inductor coils 902, including one or more intramyocardial coils, epicardial coils, and/or endocardial coils, and the exposed ends 904, 906 of each of the inductor coils 902 of the system 900 can be used to determine a particular location within the heart 100 that fibrillation or other abnormal electrical activity is occurring (e.g., the exposed ends 904, 906 can act as sensing electrodes). For example, the exposed ends 904, 906 of each of the inductor coils 902 can be used to detect abnormal rates of electrical activity in a particular area of the heart 100, such as relatively slow fibrillation or a period lacking any electrical activity.

In response to identifying a region of the heart experiencing fibrillation or other abnormal electrical activity, the energy delivered to particular inductor coil(s) 902 positioned closest to the location of fibrillation or other abnormal electrical activity can be modified in order to generate bipolar electroporation shocking vectors locally targeting the tissue at the location proximate the inductor coil 902. In some implementations, in response to the exposed ends 904, 906 detecting abnormal rates of electrical activity in a particular area of the heart 100, electroporation can be provided by be automatically (for example, via feedback system 1106) or manually (for example, via user input) controlling one or more inductor coils 902 proximate the area of tissue that was first detected as having abnormal rates of electrical activity to provide electroporation. As previously discussed, by delivering the electrical vectors of the pulsating electrical field directly through the cardiac tissue that is experiencing fibrillation, without passing electricity through other surrounding tissue of the patient 10 (e.g., surrounding skeletal muscle or nerves), pain experienced by the patient during defibrillation via electroporation is greatly reduced compared to pain experienced during traditional defibrillation.

In some implementations, if after providing targeted electroporation, the exposed ends 904, 906 of the respective inductor coil 902 detect that abnormal levels of electrical activity are still present, electroporation can be provided by additional (or all) inductor coils 902 of the system 900. In some implementations, if electroporation using all inductor coils 902 fails to terminate fibrillation or ventricular fibrillation or other malignant arrhythmia continues to be detected, a direct current shock is provided to the patient. For example, the inductor coil(s) 902 positioned within the heart 100 to deliver targeted electroporation can also be used to generate a higher energy, direct current shock to the heart 100 as a background shock. Similarly, electrodes 1102, 1104 can be used to generate a higher energy, direct current shock to the heart 100 if lower energy electroporation fails to terminate fibrillation or ventricular fibrillation or other malignant arrhythmia continues to be detected.

In some implementations, the reversible electroporation systems described herein are used in conjunction with standard cardiac defibrillators. For example, a standard cardiac defibrillator can be used as a backup system to perform defibrillation if performance of reversible electroporation using the systems described herein fails to terminate the cardiac arrhythmia.

While the systems described herein have been described as being used for performing electroporation-based cardiac defibrillation following detection of atrial or ventricular fibrillation, other therapies can be performed using the systems described herein, including electroporation therapies intended to prevent cardiac fibrillation from occurring. For example, before ventricular or atrial fibrillation occurs, other cardiac conditions, such as changes in heart rate, ventricular ectopy, non-sustained runs of ventricular tachycardia, close-coupled premature ventricular contractions (PVCs), and other changes in the nature of the signals received from the electrodes of the system (e.g., amplitude, frequency, slew, or a combination thereof) can be detected, and in response to detecting one or more of these conditions, a low intensity electroporation sequence can be preemptively delivered using the above-described electroporation systems in order to prevent the ventricular fibrillation or other forms of more malignant arrhythmia from developing. For example, as previously discussed, the electrodes 1102, 1104 positioned on one or more surfaces of the heart 100 can be used to detect and record electrical signals generated by the heart 100. The electrodes 1102, 1104 can be communicably coupled to a feedback system 1106 to transmit electrical signals generated by the heart 100 and detected by the electrodes 1102, 1104 to the feedback system 1106. The feedback system 1106 can detect various cardiac conditions based on the signals received from the electrodes 1102, 1104 and can modify the energy delivered to the electrodes 1102, 1104 based on the conditions detected. In some implementations, the electrodes 1102, 1104 are controlled to deliver electroporation based on the cardiac condition detected based on signals generated by the electrodes 1102, 1104. In some implementations, the electrodes 1102, 1104 are controlled to pace the heart in order to change the patient's heart rate based on the cardiac condition detected based on signals generated by the electrodes 1102, 1104. As described in further detail herein, in some implementations, the signals generated by the electrodes 1102, 1104 can be evaluated using a machine learning model in order to detect and predict the occurrence of various cardiac conditions and prevent fibrillation.

In some implementations, a machine learning model is implemented in order to facilitate the provision of defibrillation and other electroporation therapies using the electroporation systems described herein. For example, a continuous machine learning model can be trained to detect various cardiac conditions, such as atrial or ventricular fibrillation, using local far field electrogram data that is continuously recorded for a patient (such as the signals generated by the electrodes of the systems described herein) as training data. Based on this training data, the machine learning model can predict and detect various cardiac conditions, such as instances of fibrillation, based on signals received in real-time or near real-time from one or more electrodes of the above-described electroporation systems, and can control the above-described electroporation systems to provide a therapy, such as pacing, anti-tachycardia pacing, defibrillator shock, electroporation shock, or electroporation sequences, to address the condition and prevent fibrillation. In some implementations, the machine learning model used to detect cardiac conditions is a dynamic machine learning model that continuously receives updated signals for a particular patient to further refine the model's ability to detect pre-fibrillation conditions. For example, as additional local far field electrogram data for a patient is received from the electrodes of the electroporation system, such as signals indicating changes in heart rate, local signals, and changes in amplitude of QRS complexes, these updated signals are continuously provided to the machine learning model as input, and the machine learning model is automatically updated based on the updated electrogram data. As a result of continuously updating the machine learning model by providing updated electrogram data for the patient as input to the model, the model's ability to identify cardiac conditions, predict and detect fibrillation, and provide appropriate treatment is improved.

In some implementations, the machine learning model is patient-specific and is based on local far field electrogram data recorded for a particular patient being treated with the electroporation system. In some implementations, the machine learning model is transferrable between patients such that a model trained using data collected from a first patient can be used to predict cardiac conditions and control electroporation systems to prevent fibrillation in other patients. In some implementations, the machine learning model is trained using local far field electrogram data recorded for multiple patients in order to improve the predictive power and transferability of the machine learning model between patients.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the process depicted in the accompanying figures do not necessarily require the particular order shown, or sequential order, to achieve desirable results. In certain implementations, multitasking and parallel processing may be advantageous.

1. A method of terminating cardiac arrhythmias, the method comprising:
generating a bipolar pulsed electrical field between a first electrode positioned on a first portion of a heart of a patient and a second electrode positioned on a second portion of the heart of the patient to cause reversible electroporation of myocardial cells of the heart.

2. The method of claim 1, further comprising monitoring the electrical activity of the heart of the patient to determine a presence of abnormal cardiac activity, wherein the pulsed electrical field is generated in response to detecting the presence of the abnormal cardiac activity.

3. The method of claim 2, wherein monitoring the electrical activity of the heart of the patient to determine the presence of abnormal cardiac activity comprises monitoring the electrical activity of the heart of the patient to detect a heart arrhythmia.

4. The method of claim 1, wherein generating a pulsed electrical field comprises generating a plurality of electrical pulses between the first electrode and the second electrode with a pulse width in a range of 1 nanosecond to 300 microseconds.

5. The method of claim 4, wherein an electrical potential of the pulsed electrical field ranges from 0.2 microCoulombs to about 30 milliCoulombs.

6. The method of claim 1, wherein generating a pulsed electrical field between the first electrode positioned on the first portion of the heart of the patient and the second electrode positioned on the second portion of the heart of the patient:
porates cell membranes of myocardial cells of the heart; and
increases cardiac ion channel conduction within the myocardial cells.

7. The method of claim 1, wherein the pulsed electrical field passes through a critical mass of myocardial tissue of the heart of the patient.

8. The method of claim 1, wherein:
the first portion of the heart comprises a first portion an epicardial surface of the heart or a first portion of an endocardial surface of the heart; and
the second portion of the heart comprises a second portion an epicardial surface of the heart or a second portion of an endocardial surface of the heart.

9. The method of claim 1, wherein:
the first portion of the heart comprises an endocardial surface of an atrium of the heart of the patient;
the second portion of the heart comprises an epicardial surface of the atrium of the heart of the patient; and
wherein generating the bipolar pulsed electrical field affects atrial conduction.

10. The method of claim 1, wherein:
the first portion of the heart comprises an endocardial surface of a ventricle of the heart of the patient; and
the second portion of the heart comprises an epicardial surface of the ventricle of the heart of the patient,
wherein generating the bipolar pulsed electrical field affects ventricular conduction.

11. The method of claim 1, wherein:
the first portion of the heart comprises a first portion of a myocardium of a ventricle or an atrium of the heart of the patient; and
the second portion of the heart comprises a second portion of a myocardium of a ventricle or an atrium of the heart of the patient.

12. The method of claim 1, further comprising:
monitoring the electrical activity of the heart of the patient following the generation of the bipolar pulsed electrical field;
determining a heart arrhythmia is present; and
in response to determining a heart arrhythmia is present, generating another bipolar pulsed electrical field.

13. The method of claim 1, further comprising:
positioning the first electrode on the first portion of the heart of the patient; and
positioning the second electrode on the second portion of the heart of the patient.

14. The method of claim 13, wherein
the first electrode comprises a first portion of an inductor coil;
the second electrode comprises a second portion of the inductor coil; and
positioning the first electrode on the first portion of the heart of the patient comprises positioning the first portion of the inductor coil against the first portion of the heart; and
positioning the second electrode on the second portion of the heart of the patient comprises positioning the second portion of the inductor coil against the second portion of the heart.

15. The method of claim 13, wherein
the first electrode and the second electrode are attached to at least one mesh;
positioning the first electrode on the first portion of the heart of the patient comprises positioning the at least one mesh on a surface of the heart to position the first electrode against the first portion of the heart; and
positioning the second electrode on the second portion of the heart of the patient comprises positioning the at least one mesh on a surface of the heart to position the second electrode against the second portion of the heart.

16. The method of claim 13 wherein:
the first electrode is attached to a first mesh;
the second electrode is attached to a second mesh;
positioning the first electrode on the first portion of the heart of the patient comprises positioning the first mesh on a first surface of the heart; and
positioning the second electrode on the second portion of the heart of the patient comprises positioning the second mesh on a second surface of the heart.

17. A system comprising:
a first electrode;
a second electrode;
a generator electrically coupled to the first electrode and the second electrode, the generator being configured to provide pulsating direct current to the first electrode and the second electrode; and
at least one attachment device configured to position the first electrode at a first portion of a heart of a patient and position the second electrode at a second portion of the heart of the patient.

18. The system of claim 17, wherein:
the at least one attachment device comprises a biocompatible mesh; and
the first electrode and the second electrode are coupled to the biocompatible mesh.

19. (canceled)

20. The system of claim 17, wherein:
the attachment device comprises an inductor coil; and
the first electrode and the second electrode are formed along the inductor coil.

21. (canceled)

22. (canceled)

23. (canceled)

24. The system of claim 17, wherein the first electrode and second electrode are configured to generate a bipolar electrical field that passes through myocardial cells of the heart of the patient when the first electrode and second electrode are positioned on the heart of the patient.

25. (canceled)
